(19) United States
(12) Patent Application Publication
(10) Pub. No.: US 2022/0151497 A1
(43) Pub. Date: May 19, 2022

(54) ELECTRODE KITS FOR MEDICAL DEVICES

(71) Applicant: GE Precision Healthcare LLC, Wauwatosa, WI (US)

(72) Inventors: Ville P. Vartiovaara, Helsinki (FI); Juha P. Virtanen, Helsinki (FI)

(73) Assignee: GE Precision Healthcare LLC, Wauwatosa, WI (US)

(21) Appl. No.: 17/097,842

(22) Filed: Nov. 13, 2020

(51) Int. Cl. A61B 5/0205; A61B 5/00

(52) U.S. Cl. CPC A61B 5/0205; A61B 5/02444; A61B 5/68335

(57) ABSTRACT

An electrode kit for preparing a medical device for a patient. The electrode kit includes a central support member having a device side and a patient side with one or more perforations are defined between. A conductive material extends through the one or more perforations to couple the patient and the medical device. A device side cover is removably coupled to the device side of the central support member, where the central support member is configured to be coupled to the medical device when the device side cover is removed. A patient side cover is removably coupled to the patient side of the central support member, where the central support member is configured to couple to the medical device to the patent when the patient side cover is removed.

### FIELD

The present disclosure generally relates to electrode kits for medical devices, and more particularly to electrode kits for preparing, repairing, and/or refurbishing medical devices.

### BACKGROUND

Electrodes are commonly used in conjunction with medical devices for providing conductivity between a patient and sensor or detecting device. For example, electrodes are often connected to, or incorporated within, medical devices used for electroencephalography (EEG), electrocardiography (ECG), and electromyography (EMG), to name a few. In general, the electrodes provide a mechanism by which electrical signals in or on the body may be detected by a detection device and displayed, often after conducting conditioning and/or analysis processes, on a host device for review by a clinician. In the example of an ECG, the detecting device includes or communicates with electrodes placed on the skin in various positions to conduct electrical activity produced by the heart. The electrodes enable the detecting device to detect this electrical activity in the medical device in a manner known in the art. Similarly, electrodes connected to or integrated within an EEG machine are placed along the scalp for detecting electricity produced by the brain, or placed at areas of interest for an EMG detecting electrical activity of a muscle.

In other examples of medical devices, electrical signals may also be transmitted, via electrodes, to the patient rather than being received from the patient. For example, these electrical signals may be transmitted to stimulate muscular contraction, such as in the case of a defibrillator for shocking a patient's heart back into rhythm.

Various types, shapes, and sizes of electrodes are known in the art, including those made by GE Healthcare® (e.g., model 2106924-0011ECG Snap Electrode with Solid Hydrogel), or 3M's® Red Dot™ Diaphoretic Soft Cloth Monitoring Solid Gel Electrode. As discussed above, the electrodes may be integrated within the medical device themselves, or provided as devices that are electrically connected to medical devices.

Most modern electrodes used in medical devices include an electrode gel for facilitating the conduction of electricity between anodes and cathodes in response to electricity fluctuations present on the skin of the patient, for example. In short, metal electrodes that are covered with a poorly soluble salt of the metal (e.g., silver covered with silver-chlorine (Ag/AgCl)), in a solution containing anions of the same salt, are nonpolarisable. Applying a voltage to the electrode causes an ion exchange that reverses the current. These processes can be described as follows: Ag+Cl⁻<->AgCl+e⁻. It will be recognized that Ag refers to solid silver, Cl⁻ to a chlorine ion, AgCl to solid silver-chloride, and e⁻ to an electron.

In a typical electrode, the electrode gel is a wet gel or a solid gel. A wet gel refers to a mixture of water and agar or some other agent to make it viscous. Solid gels may also be referred to as hydrogels, which are generally a network of cross-linked polymer chains that are hydrophilic. The hydrophilic polymer chains being held together by cross links results in a three-dimensional solid. The resultant hydrogels are highly absorbent, often containing more than 90 percent water, and be made of natural or synthetic polymeric networks. In most cases, polyvinyl alcohol (PVA) is used as a chemical host. A salt, such as KCl or NaCl, is provided to make the water electrically conductive.

Typical examples of substrate materials for providing structure to the electrode include polyethylene foam or film, non-woven fabrics, polyolefin tape, or vinyl tape, for example. A skin-compatible adhesive is provided on the skin-side of the electrode substrate for adhering the device to the patient's skin. Acrylic adhesives are the most common, though other substances such as silicon or polyurethane may also be used, for example. Exemplary skin adhesives are commercially available from Elkem, Henkel, and HB Fuller.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

One embodiment of the present disclosure generally relates to an electrode kit for preparing a medical device for a patient. The electrode kit includes a central support member having a device side and a patient side with one or more perforations are defined between. A conductive material extends through the one or more perforations to couple the patient and the medical device. A device side cover is removably coupled to the device side of the central support member, where the central support member is configured to be coupled to the medical device when the device side cover is removed. A patient side cover is removably coupled to the patient side of the central support member, where the central support member is configured to couple to the medical device to the patent when the patient side cover is removed.

In certain embodiments, the device side cover is removably coupled to the central support member via a device side adhesive, and the patient side cover is removably coupled to the central support member via a patient side adhesive. In further embodiments, the patient side adhesive and the device side adhesive are different.

In certain embodiments, a stronger bond is formed between the patient side cover and the central support member than between the device side cover and the central support member.

In certain embodiments, when the device side cover is removed and the central support member is coupled to the medical device, a stronger bond is formed between the between the central support member and the medical device than between the central support member and the patient side cover.

In certain embodiments, when the device side cover is removed and the central support member is coupled to the medical device and when the patient side cover is removed and the central support member is coupled to the patient, a stronger bond is formed between the between the central support member and the medical device than between the central support member and the patient.

In certain embodiments, the device side cover has an inside that is removably coupled to the central support member, and wherein a width of the inside of the device side cover is greater than a width of the device side of the central support member. In further embodiments, the patient side cover also has an inside that is removably coupled to the central support member, and wherein a width of the inside of the patient side cover is greater than the width of the device side of the central support member and less than the width of the inside of the device side cover.

In certain embodiments, the patient side cover further comprises a release tab, and wherein the patient side cover is removable from the central support member by applying a force on the release tab in a direction opposite the central support member.

In certain embodiments, the conductive material is a solid gel, wherein the central support member is polyethylene.

In certain embodiments, the central supporting member and the conductive material each have concentric, circular cross-sections.

In certain embodiments, at least one of the device side cover and the patient side cover defines a conductor recess surrounding a base portion, wherein the base portion is configured to be removably coupled to the central support member, and wherein the conductor recess is configured to receive the conductive material extending away from the central support member therein.

In certain embodiments, the patient side connector further comprises a centrally positioned handle for applying a force in a direction opposite the central support member to remove the patient side connector therefrom.

In certain embodiments, the medical device is configured to detect at least one of a heart rate and a respiratory rate of the patient via conduction through the conductive material.

In certain embodiments, the one or more perforations is at least two perforations coupled together by the conductive material.

In certain embodiments, the medical device is a dual electrode system, wherein the conductive material is a first conductive material and the one or more perforations forms a first set of perforations, wherein the central supporting member also defines a second set of one or more perforations therethrough, further comprising a second conductive material that extends through the second set of one or more perforations, and wherein the first conductive material is not in contact with the second conductive.

In certain embodiments, the conductive material is bisected by the central support member, and wherein the conductive material extending through the one or more perforations fixes the conductive material to the central support member.

In certain embodiments, at least one of the central support member and the patient side cover includes an alignment feature for aligning the central support member to the medical device.

Another embodiment generally relates to an electrode kit for preparing a medical device for a patient. The electrode kit includes a central support member having a device side and a patient side with a plurality of perforations defined between. A conductive material extends through the plurality of perforations to couple the device side and the patient side of the central support member. A device side cover is removably coupled to the device side of the central support member via a device side adhesive, where the central support member is configured to be coupled to the medical device when the device side cover is removed. A patient side cover is removably coupled to the patient side of the central support member via a patient side adhesive different than the device side adhesive. The central support member is configured to couple to the medical device to the patent when the patient side cover is removed, where a stronger bond is formed between the patient side cover and the central support member than between the device side cover and the central support member. The device side cover is removed and the central support member is coupled to the medical device, where a stronger bond is formed between the between the central support member and the medical device than between the central support member and the patient side cover.

Various other features, objects and advantages of the disclosure will be made apparent from the following description taken together with the drawings.

### BRIEF DESCRIPTION OF THE DRAWINGS

The present disclosure is described with reference to the following Figures.

FIG. 1 depicts an exemplary medical device incorporating an embodiment of electrodes from an exemplary electrode kit according to the present disclosure, shown in conjunction with use on a patient;

FIG. 2 depicts additional embodiments of exemplary electrodes incorporated into medical devices using exemplary electrode kits according to the present disclosure;

FIG. 3 is an isometric sectional view of one embodiment of an electrode kit according to the present disclosure, such as that used to incorporate the electrodes of FIG. 1;

FIG. 4A is an exploded front view of the electrode kit shown in FIG. 3;

FIG. 4B is an exploded isometric sectional view of the electrode kit of FIG. 3;

FIG. 5 is an isometric sectional, partially exploded view of another embodiment of electrode kit according to the present disclosure, shown before being incorporated within a medical device such as that shown in FIG. 2;

FIG. 6 is an isometric sectional view depicting the electrode kit of FIG. 5 incorporated into a medical device such as that shown in FIG. 2;

FIG. 7 is a lower isometric view of the medical device of FIG. 6 with the electrode incorporated therein;

FIG. 8 is an isometric exploded view of another exemplary embodiment of an electrode kit according to the present disclosure shown before being incorporated within a medical device, now shown for incorporating two electrodes; and

FIG. 9 depicts an exemplary method using an electrode kit with a medical device according to the present disclosure.

### DETAILED DISCLOSURE

Modern medical devices often include electrodes that are integrated directly into them. This can be advantageous within the context of a single-use device, low-cost devices, devices designed to reduce user error during setup or use, and/or devices designed to have simplified workflows and reduced setup time, for example. However, with the ever-rising cost of healthcare, the inventors have recognized that there is an unmet need to be able to replace the electrodes that are incorporated within these devices.

For example, the inventors have recognized that electrode replacement would be useful to replace a defective or questionable electrode from a medical device, to refurbish a used device by providing a new electrode for subsequent use, and/or to extend the life of a device after an earlier electrode ceases to function or no longer adheres well to the patient's skin, for example. The inventors have further identified that the shelf life of the underlying medical device and the electrodes to be used therewith are often much greater when these components are kept separately than when integrated together. Therefore, a solution in which an electrode kit may be used for integrating the electrode within the medical device shortly before or at the time of use would reduce waste, and/or reduce the amount of inventory needed to keep on hand over medical devices with integrated electrodes presently known in the art.

One additional reason in which medical devices often include integrated electrodes is in the context of a device designed to provide guidance for the relative placement of these electrodes. In other words, by integrating the electrodes within a portion of the medical device itself, the entire device may be positioned on the body (e.g., relative to one or more landmarks) without necessitating the selection of placement for each separate electrode. This essentially guarantees the positioning of the electrodes relative to each other, thereby simplifying the process and improving the accuracy and/or quality of data received in the medical device.

It will be recognized that within the context of the present disclosure, electrodes are not limited to applications of detecting free electrical charges or electricity. For example, electrodes may also be used to conducting energy and signals to a patient. Moreover, electrodes need not be used for electrical conductance to and/or from a patient. For example, electrodes may be use as capacitive (non-conductive) measurement devices (e.g., using a high permittivity gel for defibrillation-proof ECG), a thermal measurement device (e.g., for an accurate thermometer patch), and/or acoustic measurement device (e.g., for ultrasound), to name a few. For the sake of simplicity, each of these applications will be referred to as the electrode simply being “conductive” in some manner.

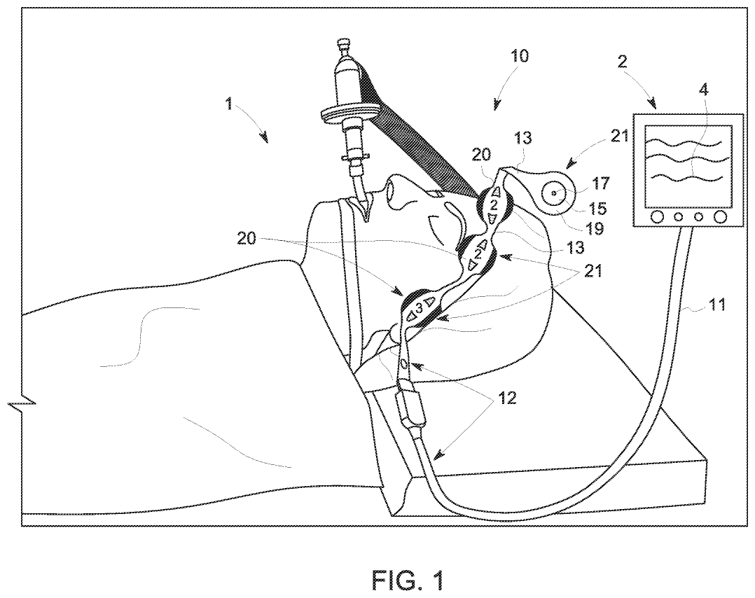

FIG. 1 depicts an exemplary medical device 10 for detecting electrical signals from a patient, in the present example is a GE Entropy™ Module produced by GE Healthcare configured for monitoring brain activity. The signals detected by the medical device 10 are then sent via a host device 2 having a display 4 for review by a clinician. The medical device 10 and host device 2 can also be referred to as an overall system 1, and in certain embodiments may be incorporated into a single unit, for example.

In the example shown, the medical device 10 is electrically coupled to the patient via three electrodes 20 (labeled 1-3) placed on the head and face of the patient. As shown, a first housing 21 configured to receive one of the electrodes 20 (here, electrode #1) is shown flipped back to reveal the underside normally adhered to the patient's skin. In contrast to the electrodes 20 labeled as #2 and #3, there is presently no electrode within the housing 21 of the electrode 20 labeled as #1. This absence of an electrode 20 may be the result of one not yet being installed according to the present disclosure before use of the device, or that an previously installed electrode 20 has been removed for replacement according to the present disclosure, for example.

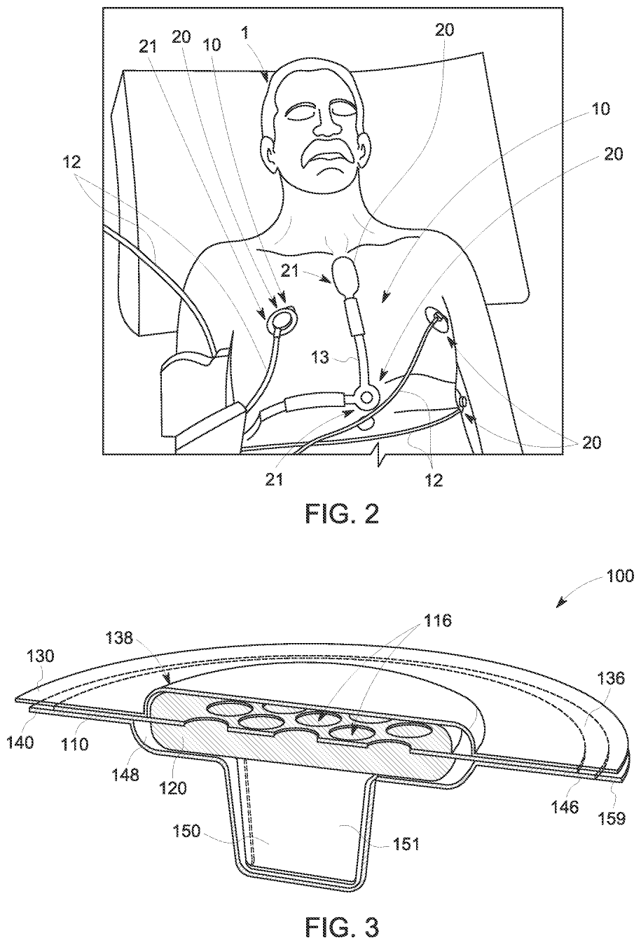

As shown in FIG. 1, the housing 21 defines an electrode cavity 15 having an electrical contact 17 therein. A surface 19 is provided surrounding the electrode cavity 15, which enables an electrode 20 according to the present disclosure to be inserted or installed within the housing 21 to thereby function in a manner similar to medical devices 10 known in the art in which the electrode 20 is permanently incorporated therein. In certain embodiments, such as shown in FIG. 2, the housing 21 may constitute its own medical device 10, rather than being one of multiple housings 21 within a common medical device 10. One example of such a medical device 10 having a single housing 21 would be a device functioning as a common (previously single-use), clip-on electrodes commonly known in the art. The medical device 10 communicates to the host device 2 via lead wires 12, including host side wires 11 and patient side wires 13.

In this regard, FIG. 2 shows multiple medical devices 10 each incorporating electrodes 20 from electrode kits 100 (see FIG. 3) according to the present disclosure. The medical devices 10 themselves may be otherwise the same as those previously known in the art, or may be particularly configured to receive electrodes via the devices and methods disclosed herein. In the example shown in FIG. 2, one medical device 10 includes two electrode housing 21 for incorporating two electrodes 20 according to the present disclosure. These two electrodes 20 are connected together via patient side wires 13 among the lead wires 12 connecting the medical device 10 to a host device 2 (FIG. 1). An additional, separate medical device 10 having a single housing 21 containing one electrode 20 according to the present disclosure, shown on the patient's right side. The second medical device 10 may be coupled to the same host device as the two electrode 20 medical device 10, or to a separate host device. Additionally, FIG. 2 depicts two electrodes 20 on the patient's left side, which in the present example are presently known single-use electrodes 20 not being separable from any housing.

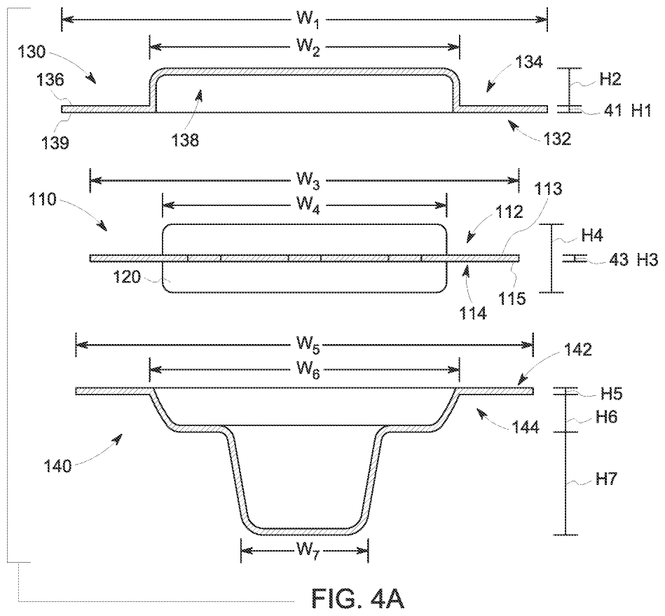

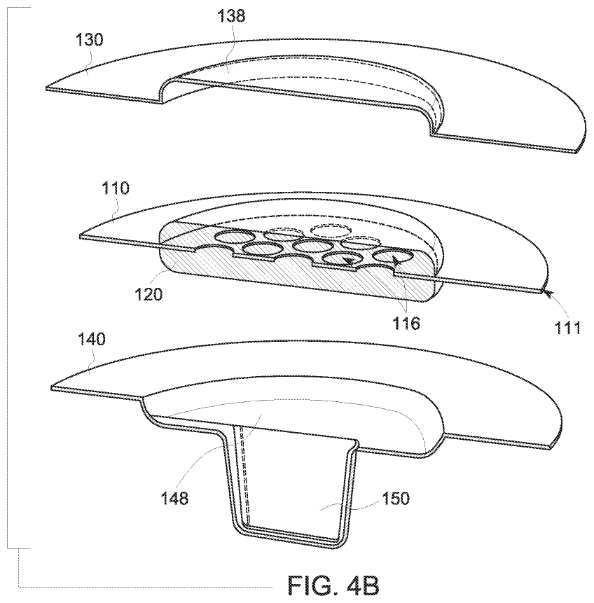

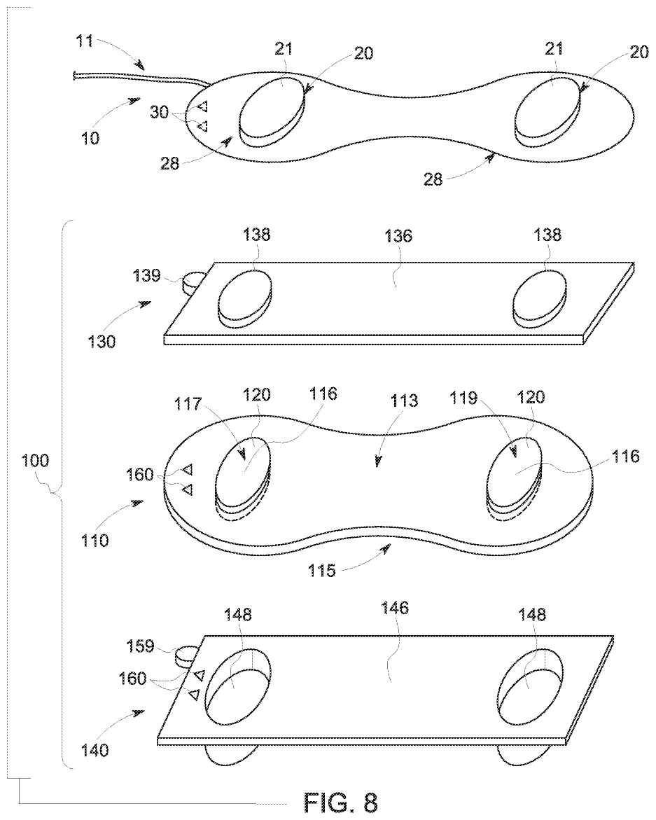

As discussed above, the inventors have recognized a need for an electrode that can be either replaced within a medical device 10, or installed within a medical device 10 in the field, for example immediately before use with a patient. FIGS. 3-4B depict an exemplary electrode kit 100 having an electrode 20 for incorporating within a medical device 10 according to the present disclosure. The electrode kit 100 includes a central support member 110 having a device side 112 and a patient side 114 opposite the device side 112. In the example shown, the central support member 110 has a substantially circular shape of uniform thickness. However, the present disclosure anticipates central support members 110 of any shape or size, such as squared, rectangular, or oblong (see e.g., FIG. 8), for example. In various embodiments, the central support member 110 has a thickness of 0.25 mm, 0.5 mm, 1.0 mm, 1.5 mm, 2.0 mm, or others, as non-limiting examples.

One or more perforations 116 are defined between the device side 112 and the patient side 114, which in the present example have a circular cross-section. However, other cross-sectional shapes for the perforations 116 are also anticipated by the present disclosure, which may be the same and/or different than the shape of the central support member 110. It will also be recognized that the perforations 116 need not have the same size of shape all the way through the central support member 110 (e.g., narrowing from the device side 112 to the patient side 114), and/or need not be the same size and/or shape as other perforations, for example. In various embodiments, the perforations have a circular shape with a diameter of 1.0 mm, 2.0 mm, 2.5 mm, 3.0 mm, 4.0 mm, 5.0 mm, and/or up to 10.0 mm or later, as non-limiting examples.

Similarly, while the present embodiment shows the perforations 116 being defined substantially near the center of the central support member 110, this is not a limitation of the present disclosure and perforations 116 may be formed anywhere (including those extended to the edge of the central support member 110).

As shown in FIGS. 3-4B, a conductive material 120 extends through the perforations 116, whereby the conduct material 120 forms a conductively pathway for electrically between the device side 112 and the patient side 114 of the central support member 110. The conductive material 120 also has a device side 122 and an opposite patient side 124 and as shown forms a generally cylindrical shape. As best shown in FIG. 4A, the central support member 110 has a height H3 defined between the device side 112 and the patient side 114, and a width W3. The central support member 110 is presently shown having a circular cross-section; though other shapes are also anticipated by the present disclosure (for example, but not limited to central support members 110 configured to support more than one conductive material 120).

Similarly, the conductive material 120 has a height H4 and an edge 111 defining a width W4. In the present example, the conductive material 120 is approximately bisected by the central support member 110, though this is not a requirement. In certain embodiments, the conductive material 120 is added to the central support member 110 via an over-molding process known in the art such that, by virtue of extending through the perforations 116, the conductive material 120 becomes fixated with the central support member 110. In the example shown, the perforations 116 prevent the conductive material 120 from moving relative to the central support member 110 not only in the up and down direction (i.e., preventing removal of the conductive material 120 away from the central support member 110), but from relative rotation therebetween due to the use of multiple perforations 116.

As shown in FIGS. 3-4B, the central support member 110 further includes a device side adhesive 113 on the device side 112, as well as a patient side adhesive 115 on the patient side 114. The device side adhesive 113 allows the conductive material 120 to be affixed to the medical device 10 via the central support member 110 by positioning the device side adhesive 113 on the surface 19 (see FIG. 1) surrounding the electrode cavity 15 of the medical device 10 to form the electrode 20. Additionally, as will be discussed further below, the device side adhesive 113 allows for retention of a device side cover 130 positioned on the device side 112 of the central support member 110 when the electrode kit 100 is in a stored state, or in other words before installation within the medical device 10. Exemplary device side adhesives include but are not limited to double coated polyethylene tape, acrylic adhesive tape, and others known in the art.

Similarly, the patient side adhesive 115 is configured to be adhered to the skin of the patient in a manner such as known for electrodes 20 presently known in the art. In other words, the patient side adhesive 115 is a skin-compatible adhesive configured to retain the electrode 20 on the skin of the patient after incorporating within the medical device 10. Exemplary patient side adhesives include but are not limited to Vancive 2120U+3M1774W by Avery Dennison® and/or NMC TA-100+3M 1522 medical tape by 3M®.

As with the device side adhesive 113, the patient side adhesive 115 is also used in the stored state of the electrode kit 100 to retain a patient side cover 140, which is removable before the medical device 10 and particularly the electrode 20 thereof is adhered to the skin of the patient.

Remaining with FIGS. 3-4B, the inventors have developed a device side cover 130 and patient side cover 140 for protecting the conductive material 120 in a safe, secure, and in certain embodiments sterile manner before installation within the medical device 10. The device side cover 130 includes an inside 132 and an outside 134 opposite the inside 132. A height H1 is defined between the inside 132 and outside 134, and the device side cover 130 has a width W1, which in the present example is circular.

The device side cover 130 includes a base portion 136, which as shown in FIG. 3 provides a basis for adhering to the central support member 110 via the device side adhesive 113 thereon. The device side cover 130 also defines a conductor recess 138 having a height H2 and width W2 configured for receiving a portion of the conductive material 120 therein when the electrode kit 100 is in a stored state with the device side cover 130 positioned on the central support member 110. As stated above, the conductive material 120 has a width W4 and a height H4. In certain embodiments, the conductor recess 130 is defined such that the height H2 is sufficiently larger than the height H4 of the conductive material 120, and likewise the width W2 is sufficiently larger than the width W4. This configuration provides that the inside 132 of the device side cover 130 does not contact the conductive material 120 when positioned on the central support member 110. By way of non-limiting example, “sufficiently” may mean 0.5 mm, 1.0 mm, 2.0 mm, 2.5 mm, 3.0 mm, 4.0 mm, 5.0 mm, or other distances between zero and 10 mm, for example, which may vary depending on the choice of materials for the conductive material 120, central support member 110, device side cover 130, and/or patient side cover 140 (discussed below), for example. However, the present disclosure also anticipates configurations in which the inside 132 of the device side cover 130 may contact the conductive material 120 at least at times.

As shown in FIGS. 3-4B, the device side cover 130 also includes a release tab 139 by which the user may grasp the device side cover 130 for removing it from the central support member 110, for example to install within a medical device 10. In the present embodiment, the release tab 139 is provided by virtue of the device side cover 130 having a width W1 that is greater than the width W3 of the central support member 110. However, other embodiments, such as that shown in FIG. 8, provide for a separate extension from the device side cover 130 as the release tab 139, for example.

The embodiment of FIGS. 3-4B further depicts a patient side cover 140 having an inside 142 and outside 144. The patient side cover 140 extends a height H5 and has a width W5. In a similar manner to the device side cover 130, the patient side cover 140 has a base portion 146, which in the present example is configured to be temporarily adhered to the central support member 110 via patient side adhesive 115 on the patient side 114 thereof.

The patient side cover 140 further defines a conductor recess 148, which has a height H6 and width W6. As with the device side cover 130, the conductor recess 148 may be configured so as to avoid contact with the conductive surface 120, or may be provided such that contact does at some or all times occur between the patient side cover 140 and the conductive material 120. Exemplary details regarding this sizing is provided in conjunction with the device side cover 130 above.

In certain embodiments, such as that shown in FIG. 8, the patient side cover 140 is also provided with a release tab 159 by which the user may grasp the patient side cover 140 for removal from the central support member 110, in the present example in preparation for adhering the patient side adhesive 115 on the patient side 114 of the central support member 110 to the patient. In other examples, such as that shown in FIGS. 3-4B, a grip region 150 extends farther from the base portion 146 than the portion defining the conductor recess 148, allowing the user to grasp the patient side cover 140 by the grip region 150. The grip region 150 extends from the portion defining the conductor recess 148 by a height H7, and has a width W7 and thickness T7. In the example shown, the grip regions 150 defines a hollow space 151 therein, which allows the user to pinch the grip region 150 for removal of the patient side cover 140. In addition to providing assistance in grasping the grip region 150, providing hollow space 151 within the grip region 150 provides that pinching of this space causes a positive pressure of the air therein, further assisting in the removal process of the patient side cover 140 from the central support member 110.

It will be recognized that a separate release tab 159 may also be provided in conjunction with a patient side cover 140 having a grip region 150, such as to break the seal during removal, particularly in cases in which the grip region 150 does not have a compressible hollow space 151 therein.

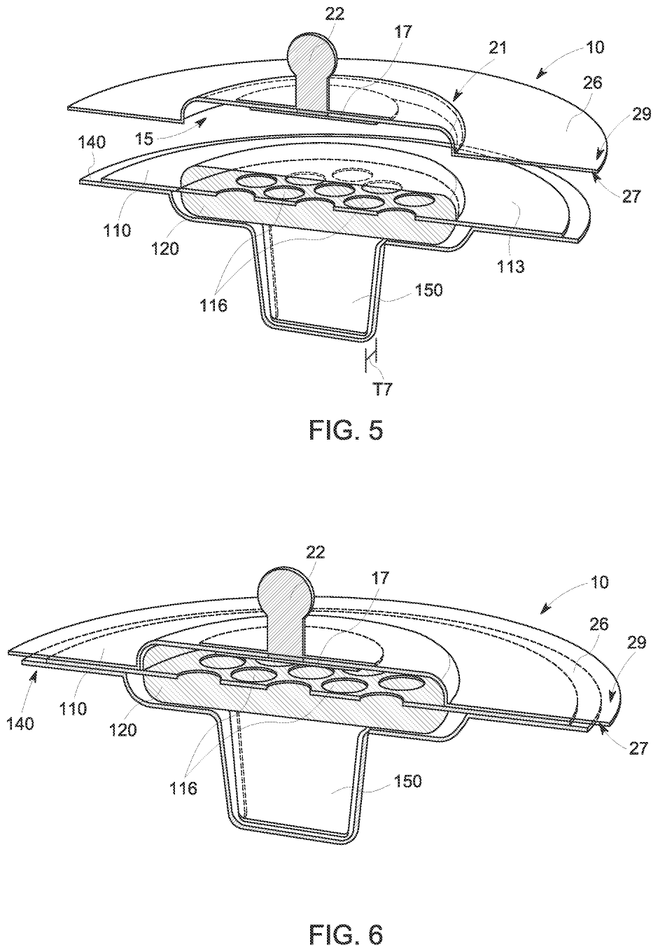

FIG. 4B depicts the device side cover 130 and patient side cover 140 removed from the central support member 110. However, in certain embodiments, the electrode kit 100 is configured in a manner such that the central support member 110 would not in normal practice be removed from both the device side cover 130 and patient side cover 140 while also not being installed within a medical device 10. For example, FIG. 5 depicts and electrode kit 100 with the device side cover 130 removed, and the central support member 110 about to be installed in the medical device 10. In the example, the medical device 10 resembles a typical single-use electrode, that is instead configured such that the medical device 10 is essentially a housing 21 for receiving the conductive material 120 according to the present disclosure. After the device side cover 130 has been removed from the central support member 110, and any previously installed conductive materials 120 (and potentially central support members 110) removed from the housing 21, the central support member 110 is adhered to an inside 27 of the housing 121 of the medical device 10 via the device side adhesive 113, as shown in FIG. 6. At this point, the patient side cover 140 may be removed, for example by grasping the grip region 150 and pulling in a downward direction, in certain embodiments also compressing the thickness T7 of the grip region 150 to remove any air from the hollow space 151 defined therein.

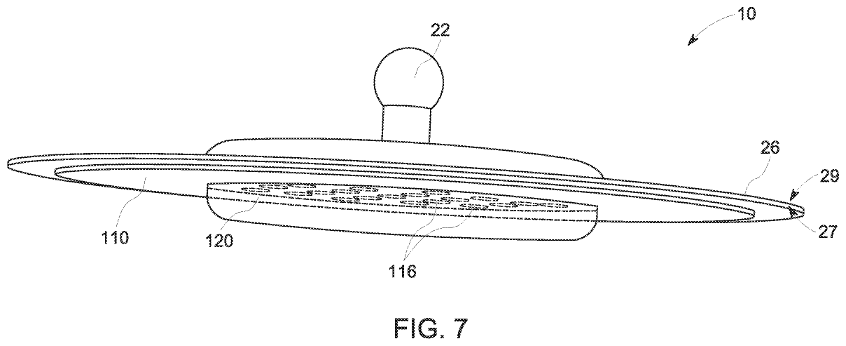

The resultant medical device 10 is shown in FIG. 7, which shows the central support member 110 adhered to the housing 21 of the medical device 10 with the patient side cover 140 removed. In certain configurations, the device side adhesive 113 is stronger (in other words, creates a stronger bond when adhered, requiring greater force to remove) than the patient side adhesive 115 such that providing a downward force on the patient side cover 140 causes the patient side cover 140 to decouple from the central support member 110, rather than the central support member 110 being decoupled from the housing 21 of the medical device 10, for example.

FIG. 8 depicts another exemplary electrode kit 100 configured for installation within a medical device 10, in this case one having housings 21 for two electrodes 20. Each of the housings 21 is defined in a similar manner to that previously described, and thus the electrode kit 100 includes a single support member 110 providing for two or more conductive materials 120. In the embodiment shown, each of the conductive materials 120 extends through a single perforation 116 defined within the central support member 110, such that the perforations 116 define a first set 117 and a second set 119. It will be recognized that the first set 117 and second set 119 may comprise more than one perforation 116 each, for example.

In the present example, a release tab 139 is provided for the device side cover 130, as well as a release tab 159 for the patient side cover 140. Additionally, alignment features 160 are provided on the medical device 10, with the corresponding alignment features 160 on the central support member 110 and/or patient side cover 140. In particular, the alignment features 160 on the central support member 110 and/or patient side cover 140 provide direction for the user to install the conductive material 120 of the electrode kit 110 within the medical device 10, such that, for example, the device is not installed in a 180° rotated configuration where this alignment is relevant to the medical device 10.

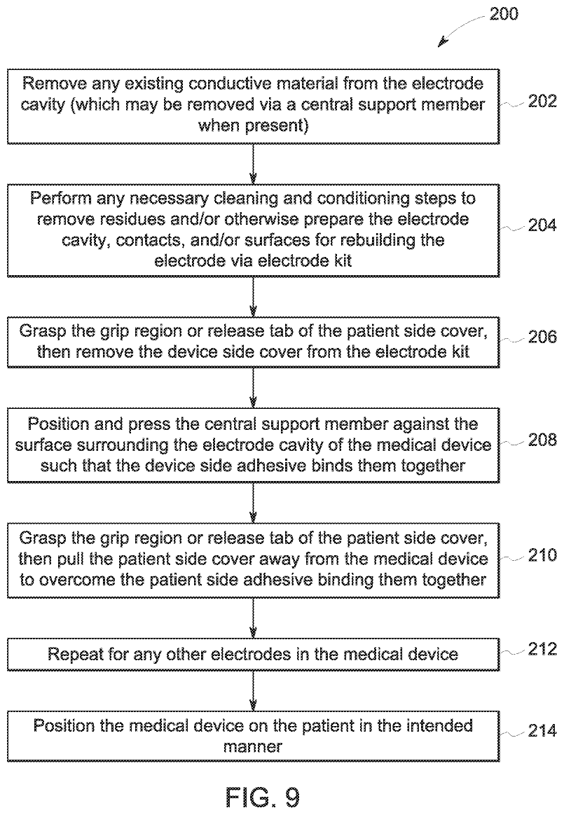

In this manner, the exemplary electrode kit 100 of FIG. 3 may be installed in a medical device 10 (or housing 21) by the following steps, as shown in the exemplary method 200 of FIG. 9:
Step 202: Removing any existing conductive material 120 from the electrode cavity 15 (potentially) removing via the central support member 110 when present)
Step 204: Performing any necessary cleaning and conditioning steps to remove residues and/or otherwise prepare the electrode cavity 15, contacts 17, and/or surfaces 19 for rebuilding the electrode 20 using an electrode kit 100 presently disclosed
Step 206: Grasping the grip region 150 of the patient side cover 140 where applicable (or elsewhere), then removing and discarding the device side cover 130 from the electrode kit 100 (using a release tab 139 where applicable)
Step 208: Positioning and pressing the central support member 110 against the surface 19 surrounding the electrode cavity 15 of the medical device 10 such that the device side adhesive 113 binds them together
Step 210: Grasping the grip region 150 of the patient side cover 140 where applicable (or elsewhere), then pulling the patient side cover 140 away from the medical device 10 to overcome the patient side adhesive 115 binding them together. The stronger bond of the device side adhesive 113 relative to the patient side adhesive 115 (when configured in this manner), ensures that the pulling force on the grip region 150 removes the patient side cover 140 from the central support member 110, rather than removing the central support member 110 from the medical device 10)
Step 212: Repeating for any other necessary electrodes 20 in the medical device
Step 214: Positioning the medical device 10 on the patient in the intended manner

This written description uses examples to disclose the invention, including the best mode, and also to enable any person skilled in the art to make and use the invention. Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

What is claimed is:

1. An electrode kit for preparing a medical device for a patient, the electrode kit comprising:
a central support member having a device side and a patient side with one or more perforations are defined between;
a conductive material that extends through the one or more perforations to couple the patient and the medical device;
a device side cover removably coupled to the device side of the central support member, wherein the central support member is configured to be coupled to the medical device when the device side cover is removed; and
a patient side cover removably coupled to the patient side of the central support member, wherein the central support member is configured to couple to the medical device to the patent when the patient side cover is removed.

2. The electrode kit according to claim 1, wherein the device side cover is removably coupled to the central support member via a device side adhesive, and wherein the patient side cover is removably coupled to the central support member via a patient side adhesive.

3. The electrode kit according to claim 2, wherein the patient side adhesive and the device side adhesive are different.

4. The electrode kit according to claim 1, wherein a stronger bond is formed between the patient side cover and the central support member than between the device side cover and the central support member.

5. The electrode kit according to claim 1, wherein when the device side cover is removed and the central support member is coupled to the medical device, a stronger bond is formed between the between the central support member and the medical device than between the central support member and the patient side cover.

6. The electrode kit according to claim 1, wherein when the device side cover is removed and the central support member is coupled to the medical device and when the patient side cover is removed and the central support member is coupled to the patient, a stronger bond is formed between the between the central support member and the medical device than between the central support member and the patient.

7. The electrode kit according to claim 1, wherein the device side cover has an inside that is removably coupled to the central support member, and wherein a width of the inside of the device side cover is greater than a width of the device side of the central support member.

8. The electrode kit according to claim 7, wherein the patient side cover also has an inside that is removably coupled to the central support member, and wherein a width of the inside of the patient side cover is greater than the width of the device side of the central support member and less than the width of the inside of the device side cover.

9. The electrode kit according to clam 1, wherein the patient side cover further comprises a release tab, and wherein the patient side cover is removable from the central support member by applying a force on the release tab in a direction opposite the central support member.

10. The electrode kit according to claim 1, wherein the conductive material is a solid gel, wherein the central support member is polyethylene.

11. The electrode kit according to claim 1, wherein the central supporting member and the conductive material each have concentric, circular cross-sections.

12. The electrode kit according to claim 1, wherein at least one of the device side cover and the patient side cover defines a conductor recess surrounding a base portion, wherein the base portion is configured to be removably coupled to the central support member, and wherein the conductor recess is configured to receive the conductive material extending away from the central support member therein.

13. The electrode kit according to claim 1, wherein the patient side connector further comprises a centrally positioned handle for applying a force in a direction opposite the central support member to remove the patient side connector therefrom.

14. The electrode kit according to claim 1, wherein the medical device is configured to detect at least one of a heart rate and a respiratory rate of the patient via conduction through the conductive material.

15. The electrode kit according to claim 1, wherein the one or more perforations is at least two perforations coupled together by the conductive material.

16. The electrode kit according to claim 1, wherein the medical device is a dual electrode system, wherein the conductive material is a first conductive material and the one or more perforations forms a first set of perforations, wherein the central supporting member also defines a second set of one or more perforations therethrough, further comprising a second conductive material that extends through the second set of one or more perforations, and wherein the first conductive material is not in contact with the second conductive material.

17. The electrode kit according to claim 1, wherein the conductive material is bisected by the central support member, and wherein the conductive material extending through the one or more perforations fixes the conductive material to the central support member.

18. The electrode kit according to claim 1, wherein at least one of the central support member and the patient side cover includes an alignment feature for aligning the central support member to the medical device.

19. An electrode kit for preparing a medical device for a patient, the electrode kit comprising:
a central support member having a device side and a patient side with a plurality of perforations defined between;
a conductive material that extends through the plurality of perforations to couple the device side and the patient side of the central support member;
a device side cover removably coupled to the device side of the central support member via a device side adhesive, wherein the central support member is configured to be coupled to the medical device when the device side cover is removed; and
a patient side cover removably coupled to the patient side of the central support member via a patient side adhesive different than the device side adhesive, wherein the central support member is configured to couple to the medical device to the patent when the patient side cover is removed;
wherein a stronger bond is formed between the patient side cover and the central support member than between the device side cover and the central support member; and
wherein when the device side cover is removed and the central support member is coupled to the medical device, a stronger bond is formed between the between the central support member and the medical device than between the central support member and the patient side cover.

20. The electrode kit according to claim 19, wherein the device side cover and the patient side cover each have an inside that is removably coupled to the central support member, wherein a width of the inside of the device side cover is greater than a width of the device side of the central support member, wherein a width of the inside of the patient side cover is greater than the width of the device side of the central support member, and wherein the width of the inside of the patient side cover is less than the width of the inside of the device side cover.
